(19) United States
(12) Patent Application Publication
(10) Pub. No.: US 2026/0158327 A1
(43) Pub. Date: Jun. 11, 2026

(54) UNIVERSAL MULTI-USE ORAL DEVICES

(71) Applicant: Jessica Cismas, Aventura, FL (US)

(72) Inventor: Jessica Cismas, Aventura, FL (US)

(21) Appl. No.: 19/417,092

(22) Filed: Dec. 11, 2025

Related U.S. Application Data
(60) Provisional application No. 63/730,816, filed on Dec. 11, 2024.

(51) Int. Cl. A63B 23/03; A61M 21/00; A61M 21/02

(52) U.S. Cl. CPC A63B 23/032; A61M 21/02; A61M 2021/0022; A61M 2210/0625

(57) ABSTRACT

Universal multi-use oral devices are disclosed. One oral device includes a body for insertion into an oral cavity, a wire connected to a point on the body and extending away and returning to couple to another point on the body apart from the point to form a shape and a space between the wire and the body, and one or more oral stimulators connected to and movable on the wire. Another oral device includes a member defining at least one space for accommodating lips or teeth of a user. The member includes an engagement wall for engagement by teeth of a user and an interior wall connected to the engagement wall. The interior wall includes an open space with at least one connection for a ball assembly located within the open space.

### REFERENCE TO RELATED APPLICATION

The present application claims priority to and the benefit of U.S. Provisional Application No. 63/730,816, filed on Dec. 11, 2024, the contents of which are incorporated herein by reference in its entirety.

### FIELD

The present disclosure relates to oral devices that serve multi purposes, such as a myofunctional therapy device, a saliva device, and an oral fidget toy.

### BACKGROUND

Oral devices can be used for any number of diagnoses such as temporomandibular joint (TMJ), attention deficit hyperactivity disorder (ADHD), focus related diagnoses, minor grinding, and jaw strength. Current oral devices require hours of customization to properly fit a user's teeth, jaw size and shape, and comfort of the user prior to use. The time required for customization of these devices can delay treatment start dates and hinder use if the device is ever lost or broken. The instant art is a novel and counterintuitive advancement in the art thereof and can provide a lower cost alternative to the current technology.

### SUMMARY

From the foregoing discussion, it should be apparent that a need exists for an apparatus, system, and method that is a one-size-fits-all multi-use oral device. Beneficially, such an apparatus, system, and method would reduce the time and expense of developing an oral device that can serve as a myofunctional therapy, a saliva device, and a novel oral fidget toy.

The subject matter of the present disclosure has been developed in response to the present state of the art, and in particular, in response to the problems and needs in the art that have not yet been fully solved by current oral devices. Accordingly, the subject matter of the present disclosure has been developed to provide apparatuses for a one-size-fits-all multi-use oral device that may overcome many or all of the above-discussed or other shortcomings in the art.

One embodiment of an oral device includes a body configured for insertion into an oral cavity of a user and a wire coupled to a point on the body and extending away from the body and returning to couple to another point on the body located apart from the first point to form a wire shape and a space between the wire and the body. The oral device further includes a set of one or more oral stimulators coupled to and movable on the wire.

Another embodiment of an oral device includes a body configured for insertion into an oral cavity of a user. The body can include a space for accommodating lips or one or more teeth of a user, an exterior wall, an engagement wall, a first side coupled to the exterior wall and configured for engagement by one or more teeth of the user, and an interior wall coupled to a second side of the engagement wall. Here, the interior wall is opposite the exterior wall, the interior wall comprises an open space, the open space comprises at least one connection for a ball assembly (e.g., an oral stimulator assembly), and the ball assembly is located within the open space of the interior wall.

Yet another embodiment of an oral device includes a member configured to define at least one space for accommodating one or more teeth of a user. Here, the member includes an engagement wall configured to be engaged by lips or one or more teeth of a user and an interior wall coupled to the engagement wall, in which the interior wall includes an open space including at least one connection for a ball assembly located within the open space of the interior wall.

Reference throughout this specification to features, advantages, or similar language does not imply that all of the features and advantages that may be realized with the subject matter of the present disclosure should be or are in any single embodiment of the subject matter. Rather, language referring to the features and advantages is understood to mean that a specific feature, advantage, or characteristic described in connection with an embodiment is included in at least one embodiment of the subject matter of the present disclosure. Thus, discussion of the features and advantages, and similar language, throughout this specification may, but do not necessarily, refer to the same embodiment.

The described features, structures, advantages, and/or characteristics of the subject matter of the present disclosure may be combined in any suitable manner in one or more embodiments and/or implementations. In the following description, numerous specific details are provided to impart a thorough understanding of embodiments of the subject matter of the present disclosure. One skilled in the relevant art will recognize that the subject matter of the present disclosure may be practiced without one or more of the specific features, details, components, materials, and/or methods of a particular embodiment or implementation. In other instances, additional features and advantages may be recognized in certain embodiments and/or implementations that may not be present in all embodiments or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims or may be learned by the practice of the subject matter as set forth hereinafter.

### BRIEF DESCRIPTION OF THE DRAWINGS

In order that the advantages of the subject matter of the present disclosure will be readily understood, a more particular description of the subject matter briefly described above will be rendered by reference to specific embodiments that are illustrated in the appended drawings. Understanding that these drawings depict only typical embodiments of the subject matter of the present disclosure and are not therefore to be considered to be limiting of its scope, the subject matter will be described and explained with additional specificity and detail through the use of the accompanying drawings, in which:

FIG. 1A is an isometric view of a multi-use oral device, according to one or more examples of the present disclosure;

FIG. 1B is the front elevation view of the multi-use oral device of FIG. 1A;

FIG. 2A is a top elevation view of a multi-use oral device, according to one or more examples of the present disclosure;

FIG. 2B is a front elevation view of the multi-use oral device of FIG. 2A;

FIG. 2C is a bottom elevation view of the multi-use oral device of FIG. 2A;

FIG. 2D is an isometric view of the multi-use oral device of FIG. 2A;

FIG. 3A is a front elevation view of a system of use of a multi-use oral device, according to one or more examples of the present disclosure;

FIG. 3B is a side view of a system of use of the multi-use oral device of FIG. 3A;

FIG. 4 is side view of an upper jaw system of use of a multi-use oral device, according to one or more examples of the present disclosure;

FIG. 5 is a side view of a lower jaw system of use of a multi-use oral device, according to one or more examples of the present disclosure;

FIG. 6A is a front view of a lower jaw system of use of a multi-use oral device, according to one or more examples of the present disclosure;

FIG. 6B is a top view of the lower jaw system of use of FIG. 6A;

FIG. 7A is a front view of multiple embodiments of multi-use oral devices, according to one or more examples of the present disclosure;

FIG. 7B is a top view of the multiple embodiments of multi-use oral devices as shown in FIG. 7A;

FIG. 7C is a rear view of the multiple embodiments of multi-use oral devices as shown in FIG. 7A;

FIG. 7D is a first angled view of the multiple embodiments of multi-use oral devices as shown in FIG. 7A;

FIG. 7E is a side view of the multiple embodiments of multi-use oral devices as shown in FIG. 7A;

FIG. 7F is a top elevated view of the multiple embodiments of multi-use oral devices as shown in FIG. 7A;

FIG. 8A is a top view of another embodiment of a multi-use oral device, according to one or more examples of the present disclosure;

FIG. 8B is a front perspective view of the multi-use oral device of FIG. 8A;

FIG. 8C is a side perspective view of the multi-use oral device of FIG. 8A;

FIG. 8D is a side view of the multi-use oral device of FIG. 8A;

FIG. 9A is a top view of the multi-use oral device of FIG. 8A showing dimensions; and

FIG. 9B is a side view of the multi-use oral device of FIG. 8A showing dimensions.

### DETAILED DESCRIPTION

Reference throughout this specification to “one embodiment,” “an embodiment,” or similar language means that a particular feature, structure, or characteristic described in connection with the embodiment is included in at least one embodiment of the subject matter of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the subject matter of the present disclosure, however, absent an express correlation to indicate otherwise, an implementation may be associated with one or more embodiments.

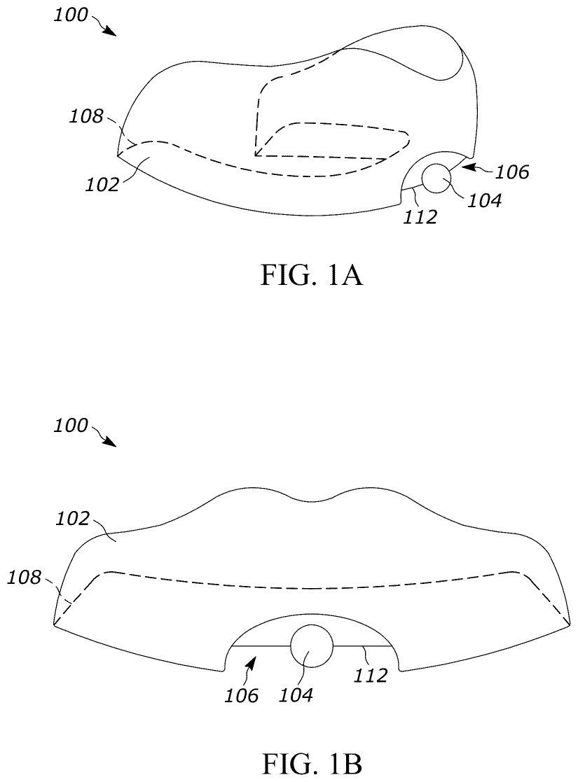

According to one example, as shown in FIGS. 1A and 1B, a one-size-fits-all multi-use oral device 100 of the present disclosure includes an exterior wall 102 and an engagement platform 108. The engagement platform 108 includes a u-shape to match the typical human bite profile, although other shapes are possible, each of which is contemplated herein. The engagement platform 108 can be a shortened u-shape and/or an extended u-shape, should most commonly be a length to cover the distance of a user's canine tooth to canine tooth. This coverage distance allows/enables for releasing muscle tension in the jaw and reprogramming muscles when a person bites on the oral device 100.

The exterior wall 102, in various embodiments, extends substantially perpendicular from the engagement platform 108. In some embodiments, the exterior wall 102 has a cupid bow shape, as shown in FIGS. 1A and 1B, to increase user comfort by including a shape compatible with the user's mouth muscles. In certain embodiments, the exterior wall 102 includes a constant height across the entire device, among other heights and/or configurations that are possible, each of which is contemplated herein.

In various embodiments, the engagement platform 108 and the exterior wall 102 are co-molded of the same material. In certain embodiments, the engagement platform 108 and exterior wall 102 are made of a hard plastic approximately 2 millimeters (mm) thick. In other embodiments, the engagement platform 108 and exterior wall 102 are made of any suitable material that is found to be safe to use in oral devices for humans.

In another example embodiment, the engagement platform 108 includes relatively thin layer that is moldable by compression on the outer sides of the platform 108. Here, the layer moldable by compression and is used to enhance user comfort by molding to the user's bite profile.

As shown in FIGS. 1A and 1B, a wall opening 106 in the exterior wall 102 defines a space for a wire 112 and a ball assembly 104 (or oral stimulator assembly). At least in the embodiment shown in FIGS. 1A and 1B, the wall opening 106 is substantially centered in the oral device 100. In other embodiments, the wall opening 106 is offset from center.

In various embodiments, the wall opening 106 is approximately 15 mm in length and 6 mm wide to form an oval shape or a substantially oval shape. In some embodiments, the wire 112 is relatively thin as to not interfere with the movement of the ball assembly 104. In certain embodiments, the wire 112 includes a dental wire, such as the wires used in orthodontic braces, among other wires and/or types of wires that are possible, each of which is contemplated herein. In some embodiments, the wire 112 is coated with a thin plastic to remove the inherent sharpness of a wire.

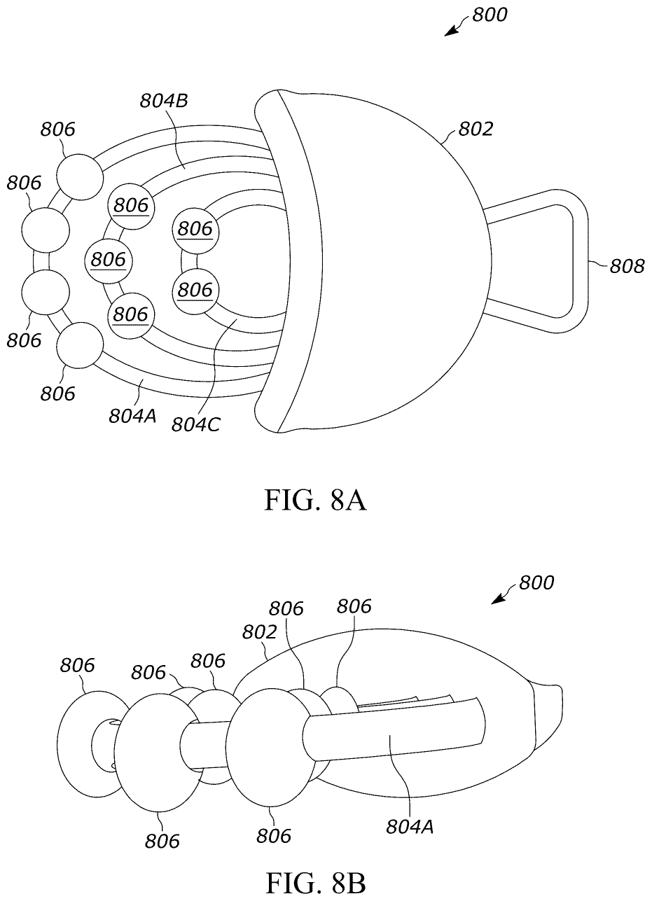

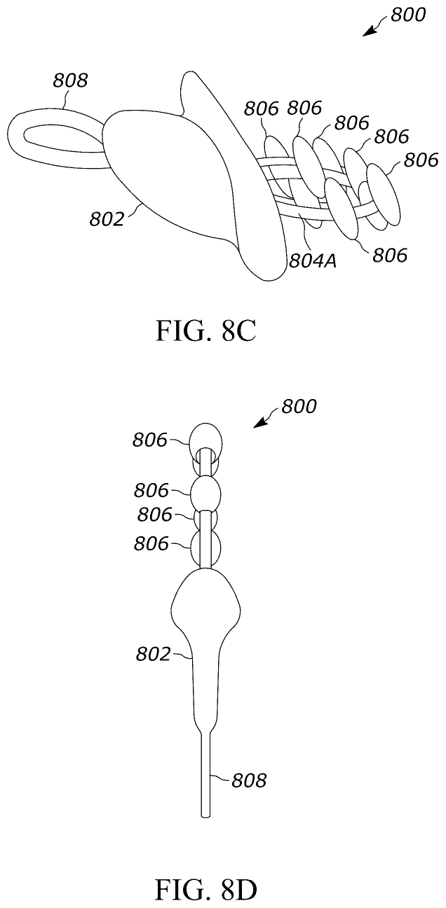

In various embodiments, the ball assembly 104 includes a singular ball (or stimulator) movable along the wire 112 within the wall opening 106. In some embodiments, the ball assembly 104 includes more than one ball, or sphere, (see, e.g., an oral stimulator 806 in FIGS. 8A through 8D) along the wire 112. In other embodiments, the ball assembly 104 includes multiple balls (or oral stimulators 806) of the same or differing sizes along the wire 112. In yet other example embodiments, the ball assembly 104 includes an oblong bead rather than a sphere.

In certain embodiments, the ball assembly 104 includes a metal sphere approximately 4 mm in diameter. In some embodiments, the ball assembly 104 includes a plastic sphere. In yet other example embodiments, the ball assembly 104 includes made of an edible material, which can include any suitable edible material that is known or developed in the future.

In various embodiments, the wire 112 and ball assembly 104 are selectively removable from the wall opening 106. The wire 112 and ball assembly 104 may be interchanged for thicker wires, adding or removing spheres or beads, or replacing damaged wires and/or ball assemblies. Additional ball assembly 104 embodiments may include adding different textures to the sphere or bead of the ball assembly 104.

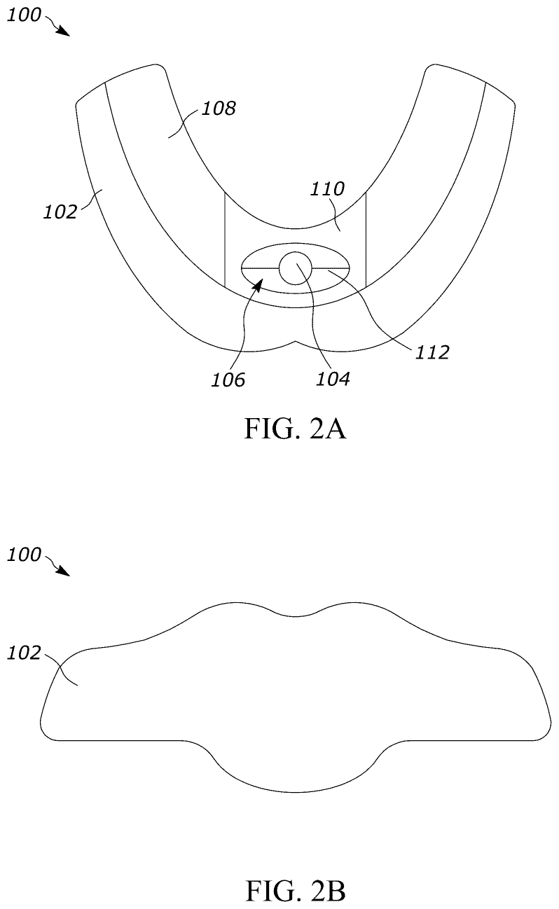

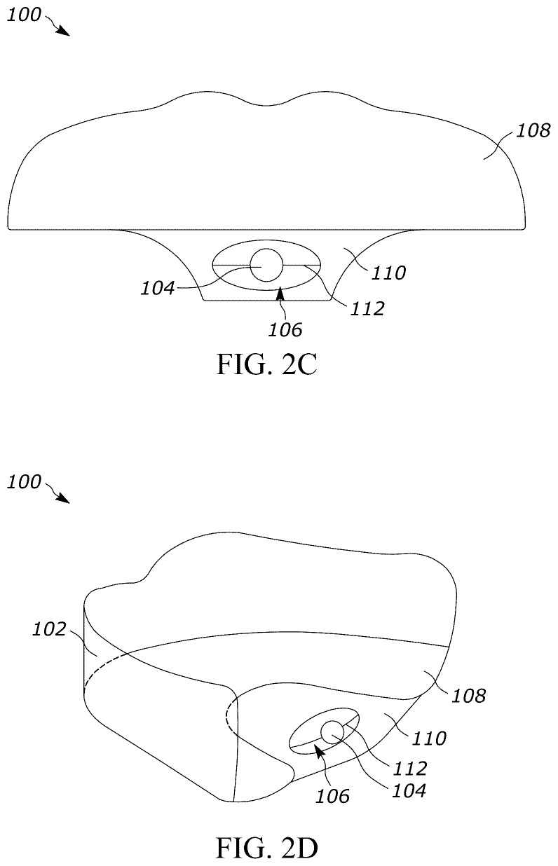

FIGS. 2A through 2D illustrate another embodiment of a one-size-fits-all multi-use device 100. The oral device 100 of FIGS. 2A through 2D includes an engagement platform 108, an exterior wall 102, and an interior wall 110. The interior wall 110 extends from the engagement platform 108 opposite the exterior wall 102.

In some embodiments, the interior wall 110 extends from the engagement platform 108 at angle, which can include any suitable angle that is known or developed in the future. In certain embodiments, the angle of extension of the interior wall 110 is approximately 45 degrees, among other angles that are possible, each of which is contemplated herein.

In some embodiments, the interior wall 108 extends from the engagement platform 108 in a direction opposite the exterior wall. For example, as shown in FIGS. 2A through 2D, the exterior wall 102 extends upward from the engagement platform 108, whereas the interior wall 110 extends downward from the engagement platform 108. In other embodiments, the exterior wall 102 and the interior wall 110 both extend upward from the engagement platform 108.

In various embodiments, the interior wall 110 is smaller and/or shorter than the exterior wall 102. In some embodiments, the interior wall 110 includes the wall opening 106, as shown in FIGS. 2A, 2C, and 2D. The wall opening 106 can define a space for the wire 112 and a ball assembly 104.

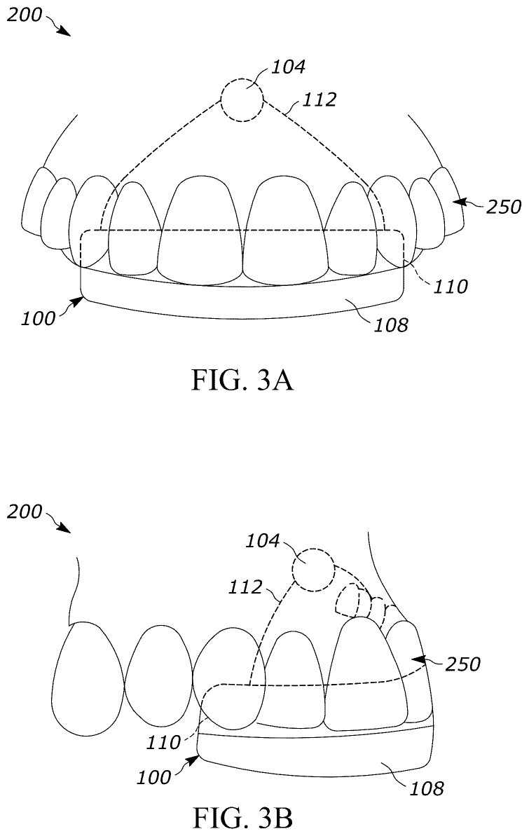

FIGS. 3A and 3B illustrate an embodiment of a system 200 of use of a one-size-fits-all oral device 100. According to at least one example of use, the system 200 includes an upper jaw 250, and corresponding lips or upper teeth, and a device 100. The device 100 includes an engagement platform 108 and an interior wall 110. The interior wall 110 matches the interior profile of the upper jaw 250. Extending from the interior wall 110 is the wire 112 and ball assembly 104. The wire 112 and ball assembly 104 extend into the upper palate of the upper jaw 250. In some examples, as shown in FIGS. 3A and 3B, a wall opening 106 is not required provided the wire 112 and ball assembly 104 are able to operate within the user's mouth. Though not shown, in the system 200 the user's outer lips provide additional retention to hold the oral device 100 in place, while the tongue 254 moves the ball assembly 104 from side to side and/or rotates the ball assembly 104 around the wire 112.

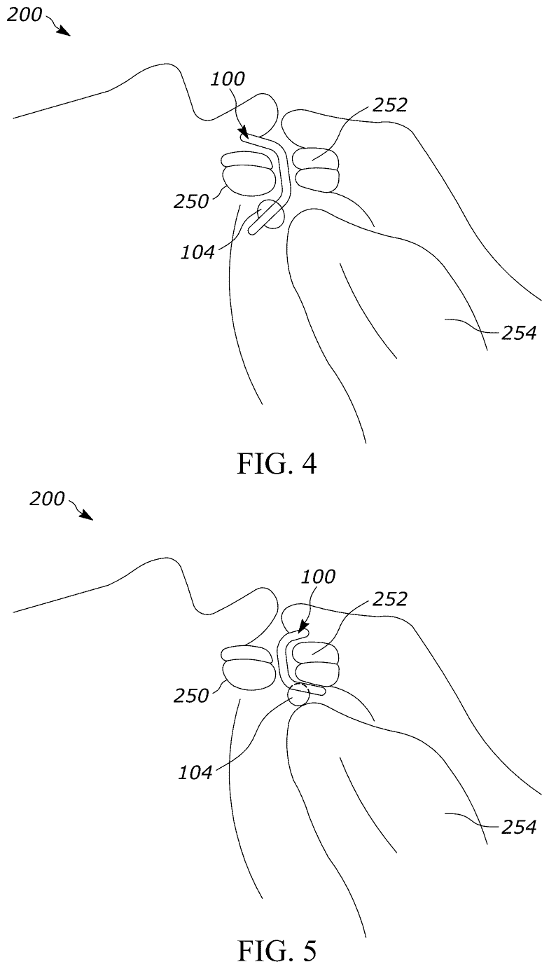

FIG. 4 illustrates an exemplary system 200 of use in which the device 100 is located around a user's upper jaw 250. As shown in FIG. 4, the user's tongue 254 interacts with the ball assembly 104. The interaction between the tongue 254 and the ball assembly 104 can have many benefits. Example benefits include, but are not limited to, increased muscle strength in the mouth and jaw, increased saliva production for dry mouth, and improved focus for ADHD (and/or other focus deficits), etc., among other benefits that are possible, each of which are contemplated herein.

FIG. 5 illustrates another exemplary system 200 of use in which the device 100 is located around a user's lower jaw 252. The oral device 100 may be used in either location based on the user's need and/or overall comfort.

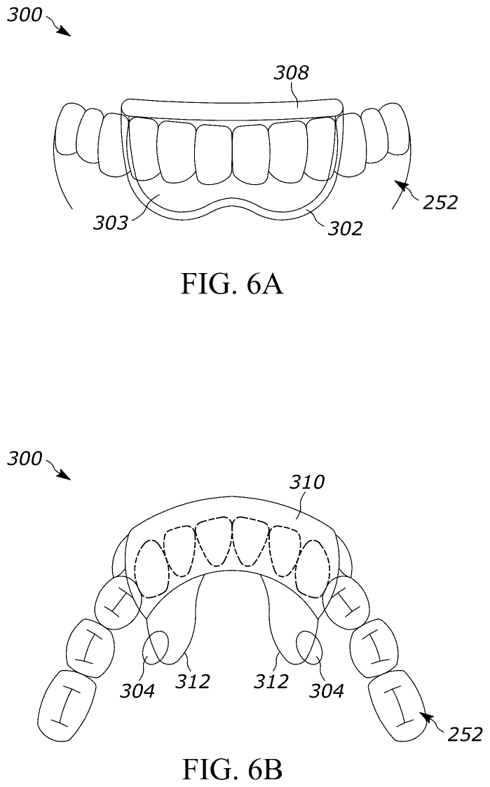

FIGS. 6A and 6B illustrate yet another embodiments of a one-size-fits-all oral device 300 of the present disclosure. The features of the oral device 300 directly correlate to the features of the oral device 100 but in a different series e.g., 300-series instead of the 100 series. Accordingly, the features of the oral device 300 are the same as or similar to the features of the oral device 100 and correspond accordingly (e.g., the wire 312 is substantially the same as the wire 112, and the ball assembly 304 is substantially the same as the ball assembly 104).

In various embodiments, the oral device 300 includes an engagement platform 308, an exterior wall 302, an interior wall 310, two wires 312, and two ball assemblies 304. Also included for reference and orientation is a lower jaw 252.

In one embodiment, the exterior wall 302 is not one solid piece but rather has an open space 303. The open space 303 may include any suitable size and/or suitable shape that is known or developed in the future.

As shown in FIG. 6A, the open space 303 matches the overall profile of the exterior wall 302. In other embodiments, the open space 303 may be a series of holes spread across the exterior wall 302. As shown in FIG. 6B, the oral device 300 may include two wires 312 and ball assemblies 304.

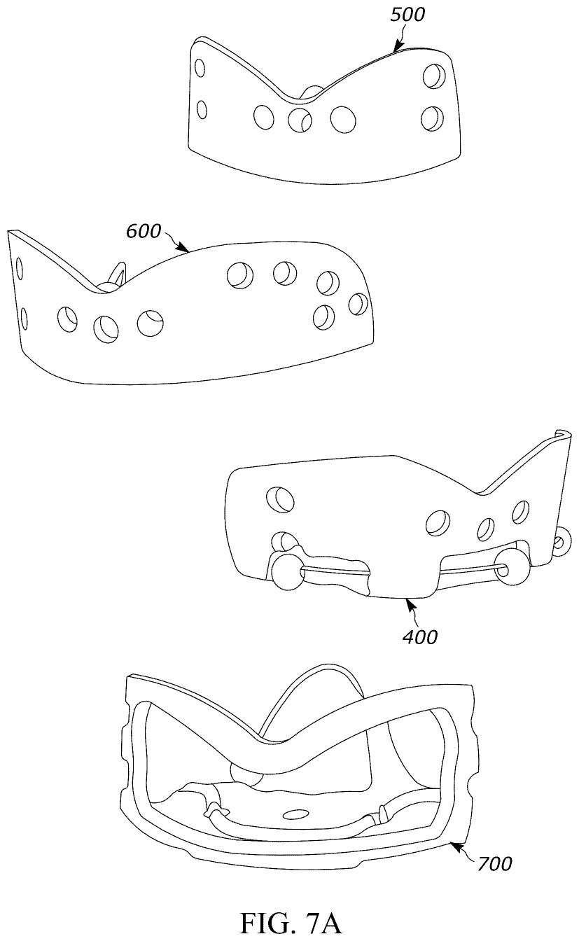

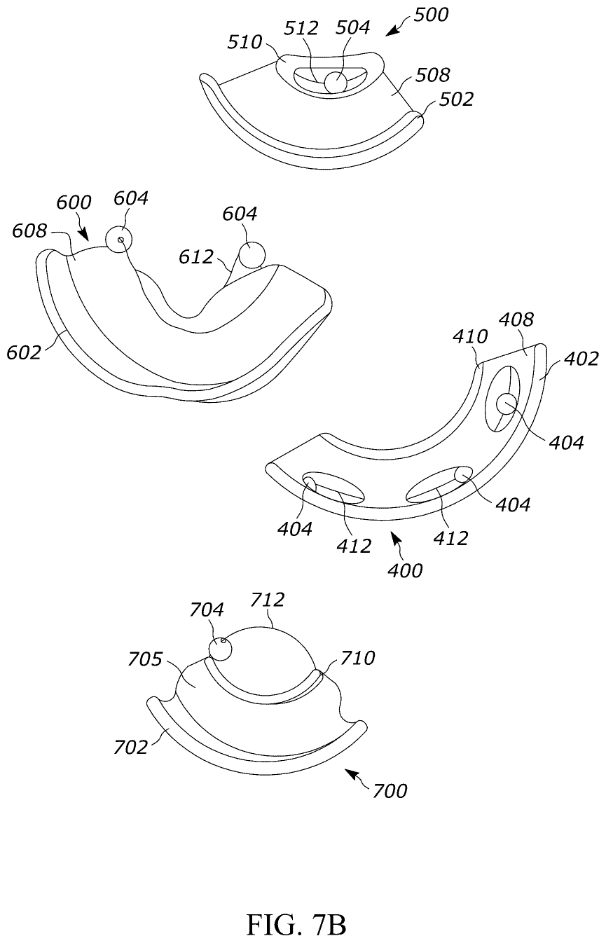

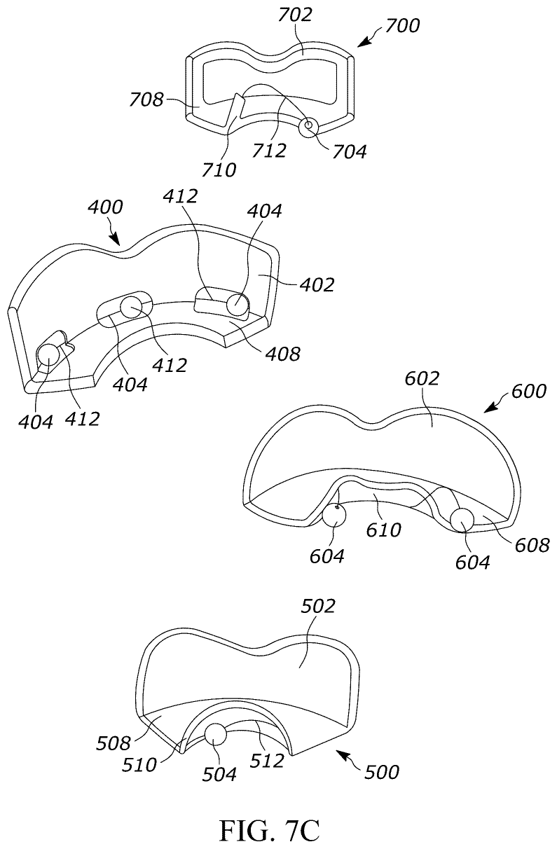

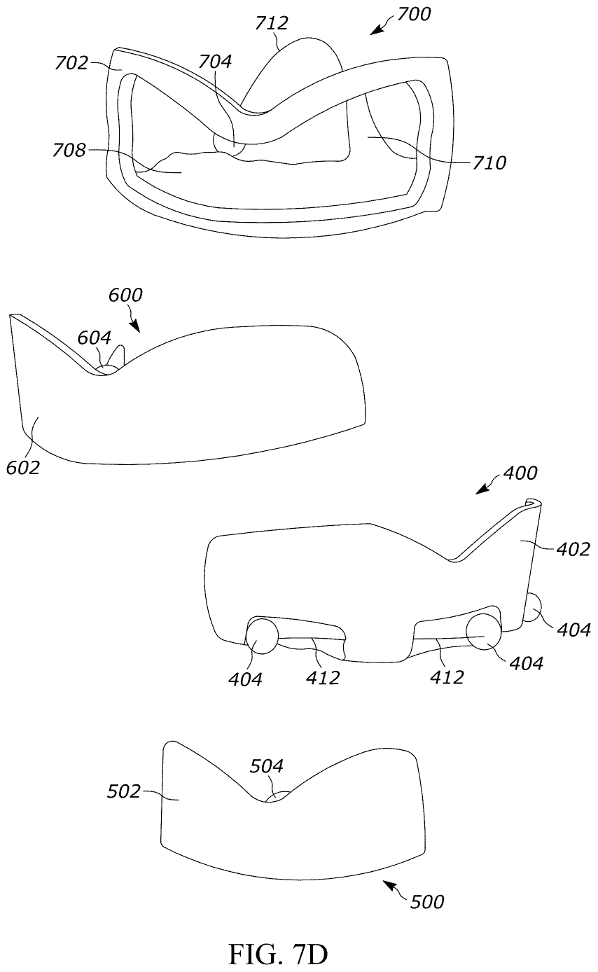

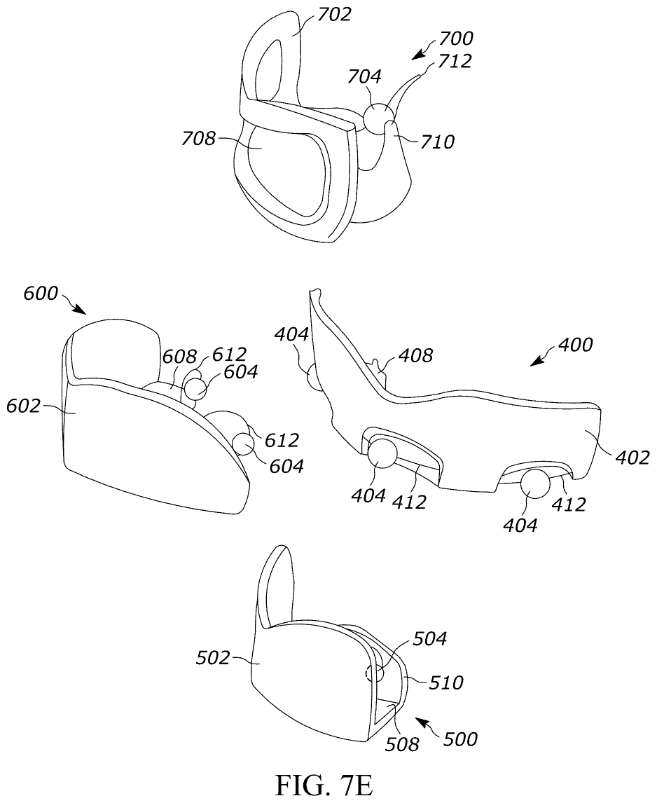

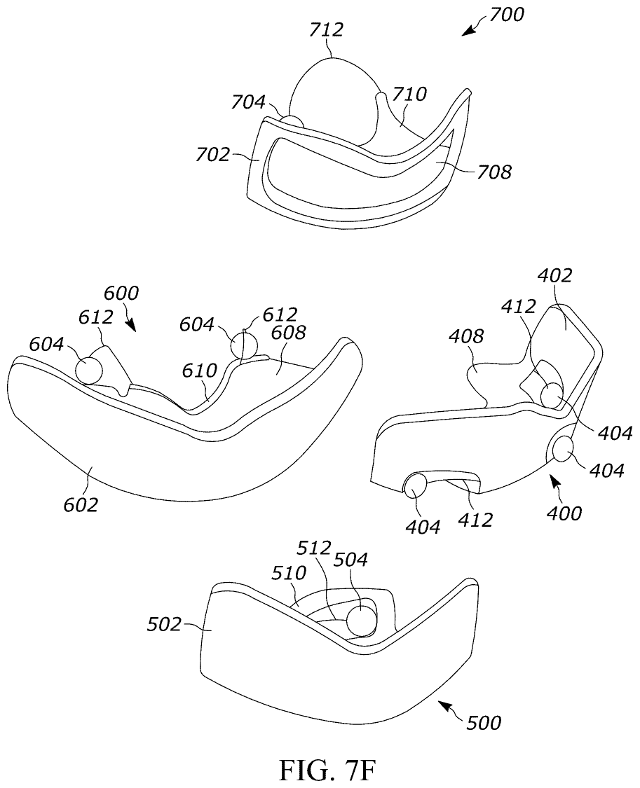

FIGS. 7A and 7F show additional examples of oral devices 400, 500, 600, and 700. The features of the oral devices 400, 500, 600, and 700 directly correlate to the features of the oral device 100 but in a different series e.g., 400-series, 500- series, 600- series, and 700- series instead of the 100 series. Accordingly, the features of the oral devices 400, 500, 600, and 700 are the same as or similar to the features of the oral device 100 and correspond accordingly (e.g., exterior walls 402, 502, 602, and 702 are substantially the same as the exterior wall 102). The oral devices 400, 500, 600, and 700 illustrate various examples of the present disclosure including different feature orientations and shapes.

An exemplary purpose of the oral device 100 includes serving as a myofunctional therapy device (e.g., a device to exercise the tongue prior to frenectomy, maxillary expansion, and development of a larger airway). Another use of the oral device 100 is as a novel oral fidget toy. That is, the oral device 100 can be used to relieve stress and increase focal points for ADHD or anyone that wants/desires to change a (bad) habit, or needs a distractions, as opposed to, for example, biting ones nails and/or cheek. In some embodiments, the oral device 100 can eliminate or at least reduce bad breath (e.g., halitosis).

Another purpose of the oral device 100 includes stimulating the production of saliva for people that have dry mouth syndrome. Yet another example of use of this oral device 100 includes as a device to treat most pains associated with TMJ. For example, if a person is using the oral device 100 for TMJ pain, the user should consult a dentist or other medical professional to determine whether a more custom fit is required (e.g., such as extending the device floor) to prevent shifting of the teeth. The various embodiments discussed herein can be a lower cost alternative to the current technology.

The oral device 100 is not meant to be worn at night, while eating or drinking. The oral device 100 is designed to be cleaned after use with soap or a non-abrasive toothpaste. The oral device 100 is not designed for placement in a dishwasher or in very hot liquids. Prior to using the oral device 100, the user should have a clean mouth, bushed and flossed, since food particles can stick to the oral device 100.

With reference to FIGS. 8A through 8D, another embodiment of an oral device 800 is illustrated. At least in the illustrated embodiment, oral device 800 includes, among other components, a body 802, a set of wires 804A, 804B, and 804C (also simply be referred to individually or collectively as, wire(s) 804), a set of oral stimulators 806 on the wire(s) 804, and a handle 808.

The body 802 may be formed of and/or include any suitable material(s) that is/are known or developed in the future capable of forming an engagement platform similar to the engagement platform 108 discussed elsewhere herein. That is, the body 802 may be formed of and/or include any suitable material or combination of materials that is capable of being engaged by the lips and/or one or more teeth of a user when inserted into the oral cavity (mouth) of the user. Example materials include, but are not limited to, a polymer, a plastic, a metal (e.g., stainless steel, a precious metal, a base metal, etc.), and/or an alloy, among other materials or combination of materials that is/are possible, each of which is contemplated herein.

Further, the body 802 may form and/or include any suitable shape(s) that is/are known or developed in the future capable of forming an engagement platform similar to the engagement platform 108 discussed elsewhere herein. That is, the body 802 may formed and/or include any suitable shape or combination of shapes that is capable of being engaged by the lips and/or one or more teeth of a user when inserted into the oral cavity (mouth) of the user. Example shapes include, but are not limited to, an irregular shape (see, e.g., FIG. 8A, etc.) or a regular shape (e.g., a sphere, an ovoid, an ellipsoid, a tetrahedron, a pyramid, a cube, and/or a cuboid, etc.), among other shapes or combination of shapes that is/are possible, each of which is contemplated herein.

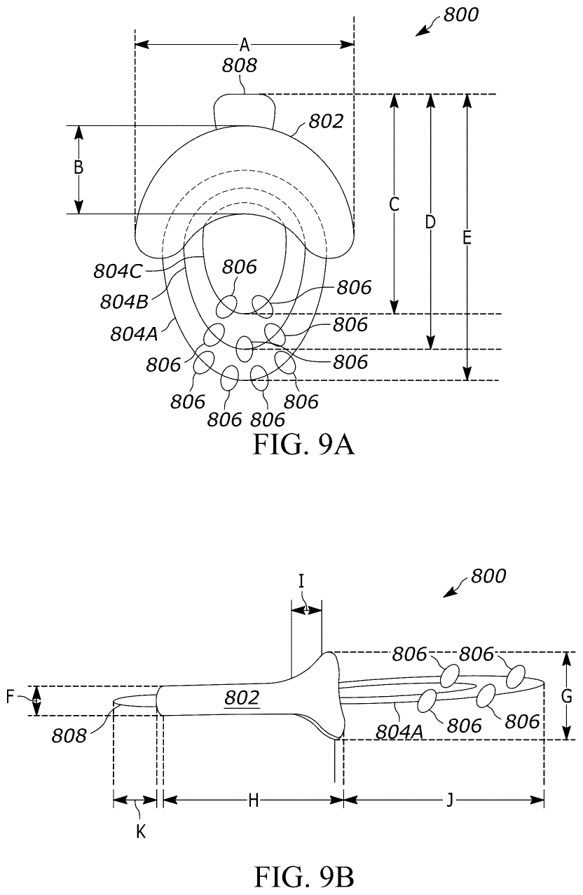

As discussed below in greater detail with reference to FIG. 9A, the body 802 may include any suitable size(s) and/or dimension(s) that is/are known or developed in the future capable of forming an engagement platform similar to the engagement platform 108 discussed elsewhere herein. That is, the body 802 may include any suitable size, combination of sizes, dimension(s), and/or combination of dimensions that is capable of being engaged by the lips and/or one or more teeth of a user when inserted into the oral cavity (mouth) of the user.

A set of wires 804 may include any suitable quantity of wires 804 greater than or equal to one (1) wire 804. That is, some embodiments of the oral device 800 include a single wire 804A, while another embodiment of the oral device 800 may include a pair of wires 804A, 804B. At least in the illustrated embodiment, the oral device 800 includes a trio of wires 804A, wire 804B, and 804C. Other embodiments of the oral device 800 may include any other possible quantity of wires 804 greater than three (3) wires 804 and each of such quantities is contemplated herein.

In various embodiments, a wire 804 includes a dental wire, such as the wires used in orthodontic braces, among other wires and/or types of wires that are possible, each of which is contemplated herein. In some embodiments, the wire 804 is coated with a thin plastic to remove the inherent sharpness of a wire.

Each wire 804 is coupled/connected to two opposing points on the body 804 thereby providing a shape to each wire 804. At least in the illustrated embodiment, each wire 804 forms a circular shape and/or u-shape that matches or substantially matches the shape of the oral cavity of a human, among other shapes for a wire 804 that are possible, each of which is contemplated herein.

In various embodiments, each wire 804 includes a set of oral stimulators 806 coupled to and/or connected to the wire. In some embodiments, a wire 804 may not include any oral stimulators 804 (e.g., zero (0) oral stimulators 804); however, this will typically be included in embodiments including a plurality of wires 804.

An oral stimulator 806 may include any suitable device(s) and/or apparatus(es) that is/are known or developed in the future capable of, among other benefits, a.) stimulating saliva production; b.) forming an oral fidget toy; c.) aligning a user's jaw; and/or d.) reducing/eliminating bad breath when engaged by the tongue of a user. An oral stimulator may be formed of and/or include any suitable material or combination of materials that is/are known or developed in the future capable of functioning as an oral stimulator 806, as described herein. Example materials include, but are not limited to, a plastic, a polymer, a ceramic, stone, wood, graphite, a metal, an alloy, stainless steel, a precious metal (e.g., gold, silver, platinum, etc.), and/or a base metal (e.g., copper, zinc, aluminum, tin, etc.), etc.), among other materials or combinations of materials that are possible, each of which is contemplated herein. In certain embodiments, the oral stimulator 806 is formed of and/or includes one or more precious metals including, for example, platinum, gold and/or silver, among other precious metals, among other precious metals that are possible, each of which is contemplated herein.

An oral stimulator 806 may include any suitable shape or combination of three-dimensional (3-D) shapes that is/are known or developed in the future capable of functioning as an oral stimulator 806, as described herein. Example shapes include, but are not limited to, a regular shape (e.g., a ball, a sphere, an ovoid, an ellipsoid, a tetrahedron, a pyramid, a cube, and/or a cuboid, etc.) or an irregular shape (e.g., an oblong bead), among other shapes or combination of shapes that is/are possible, each of which is contemplated herein.

At least in the embodiment illustrated in FIGS. 8A through 8D, an oral stimulator 806 includes a ball or sphere including a hole or aperture capable of having a wire 804 running all the way therethrough, among other shapes capable of moving via a user's tongue and/or lips engaging the oral stimulator 806 that are possible, each of which is contemplated herein. In some embodiments, an oral stimulator 806 is capable moving (e.g., sliding) along the entire length or at least a portion of the length of a wire 804 in response to a user's tongue engaging and/or applying a force to the oral stimulator 806 that causes the oral stimulator 806 to move/slide along the wire 804. In additional or alternative embodiments, an oral stimulator 806 is capable rotating around the wire 804 in response to a user's tongue engaging and/or applying a force to the oral stimulator 806 that causes the oral stimulator 806 to rotate around the wire 804. That is, various embodiments of an oral stimulator 806 are capable of sliding along and/or rotating around the wire 804. In other words, an oral stimulator 806 is capable of rotating around the wire 804 as the oral stimulator 806 is sliding along a length of the wire 804. The ability of an oral stimulator 806 to slide along the wire 804 and/or rotate around the wire 804 enables/allows, among other benefits, a.) saliva production to be stimulated in the user's oral cavity; b.) the oral stimulator 806 to function as an oral fidget toy; c.) a user's jaw to become aligned or at least more aligned; and/or d.) causes bad breath to be eliminated or at least reduced in response to the oral stimulator 806 being engaged by the tongue of a user.

An oral stimulator 806 may include any suitable size(s) and/or dimension(s) that is/are known or developed in the future capable of fitting within the oral cavity of a user. That is, an oral stimulator 806 may include any suitable size, combination of sizes, dimension(s), and/or combination of dimensions that is capable of being engaged by the tongue of a user when inserted into the oral cavity of the user.

In various embodiments, the oral stimulator 806 includes a size (e.g., height and/or width) in the range of about 2 mm to about 4 mm among other sizes/volumes that are possible, each of which is contemplated herein. In some embodiments, the oral stimulator 806 includes a size (e.g., volume) in the range of about 1.0 mm³ to about 1000 mm³, among other sizes/volumes that are possible, each of which is contemplated herein. In certain embodiments, the oral stimulator 806 includes a size/volume of about 100 mm³, among other sizes/volumes that are greater than 100 mm³ or less than 100 mm³ that are possible, each of which is contemplated herein. In each embodiment, the aperture is positioned/located at the approximate midpoint of the oral stimulator 806.

In some embodiments, an oral stimulator 806 may include a flavoring (or flavor). The flavoring may include any suitable flavoring that is known or developed in the future. Example flavorings include, but are not limited to, a fruit flavoring (e.g., apple, strawberry, raspberry, pear, blackberry, blueberry, peach, plum, apricot, banana, pineapple, watermelon, grape, cherry, mango, papaya, kiwi, cantaloupe, orange, clementine, lemon, grapefruit, and/or lime, etc.), a mint flavoring (e.g., spearmint, peppermint, etc.), a chocolate flavoring, a coffee flavoring, and/or a vanilla flavoring, etc., among other flavorings/flavors that are possible, each of which is contemplated herein.

A set of oral stimulators 806 on a particular wire 804 may include any suitable quantity of oral stimulators 806 that is known or developed in the future greater than or equal to one (1) oral stimulator 806. For example, a wire 804 may include one (1) oral stimulator 806, two (2) oral stimulators 806, three (3) oral stimulators 806, four (4) oral stimulators 806, five (5) oral stimulators 806, . . . ten (10) oral stimulators 806, etc., among other quantities of oral stimulators greater than 5 oral stimulators 806 or 10 oral stimulators 806 that are possible, each of which is contemplated herein.

In some embodiments that include more than one oral stimulator 806 on a wire 804, each (e.g., all) oral stimulator 806 includes the same shape. In other embodiments, each oral stimulator 806 includes a different shape. In still other embodiments, at least two (2) oral stimulators 806 include the same shape or at least two oral stimulators 806 include different shapes.

In additional or alternative embodiments that include more than one oral stimulator 806 on a wire 804, each oral stimulator 806 includes the same size and/or dimensions. In other embodiments, each oral stimulator 806 includes a different size and/or dimensions. In still other embodiments, at least two oral stimulators 806 include the same size and/or dimensions or at least two oral stimulators 806 include different sizes and/or dimensions.

In other additional or alternative embodiments that include more than one oral stimulator 806 on a wire 804, each oral stimulator 806 is formed of and/or includes the same material. In other embodiments, each oral stimulator 806 formed of and/or includes a different material. In still other embodiments, at least two oral stimulators 806 are formed of and/or include the same material or at least two oral stimulators 806 formed of and/or include a different material.

In some embodiments that include at least two (2) wires 804, each oral stimulator 806 on the two wires 804 includes the same type of shape. For example, a first wire 804 and a second wire 804 may each include one or more oral stimulators 806 including a ball, among other shapes that are possible, each of which is contemplated herein.

In other embodiments, the oral stimulator(s) 806 on the two wires 804 includes a different type of shape. For example, each oral stimulator 806 on a first wire 804 may include a sphere and each oral stimulator 806 on a second wire 804 may include a cube, among other shapes that are possible, each of which is contemplated herein.

In still other embodiments, at least one oral stimulator 806 on a first wire 804 includes the same shape as at least one oral stimulator on a second wire 804 or at least one oral stimulator 806 on a first wire 804 includes a different shape than at least one oral stimulator on a second wire 804. For example, at least one oral stimulator 806 on a first wire 804 and at least one oral stimulator 806 on a second wire 804 may each include a sphere and/or at least one oral stimulator 806 on a first wire 804 includes a sphere and at least one oral stimulator 806 on a second wire 804 may include a cube, among other shapes that are possible, each of which is contemplated herein.

In additional or alternative embodiments that include at least two wires 804, each oral stimulator 806 on the two wires 804 includes the same size and/or dimensions. In other embodiments, the oral stimulator(s) 806 on each of the two wires 804 includes a different size and/or dimensions. For example, each oral stimulator 806 on a first wire 804 may be larger than each oral stimulator on a second wire 804, or vice versa. In another example, the oral stimulator(s) 806 on a wire 804 closest to the body 802 (e.g., wire 804C) may include a larger or smaller size than the oral stimulator(s) 806 on a wire 804 farther from the body 802 (e.g., wire 804A or wire 804B).

In still other embodiments, at least one oral stimulator 806 on a first wire 804 includes the same size and/or dimensions as at least one oral stimulator on a second wire 804 or at least one oral stimulator 806 on a first wire 804 includes a different size and/or dimensions than at least one oral stimulator on a second wire 804. For example, at least one oral stimulator on a first wire 804 may be larger than at least one oral stimulator on a second wire 804, or vice versa. In a further example, at least one oral stimulator 806 on a wire 804 closest to the body 802 (e.g., wire 804C) may include a larger or smaller size than at least one oral stimulator 806 on a wire 804 farther away from the body 802 (e.g., wire 804A or wire 804B).

In further additional or alternative embodiments that include at least two wires 804, each oral stimulator 806 on the two wires 804 is formed of and/or includes the same material. For example, a first wire 804 and a second wire 804 may each include one or more oral stimulators 806 including silver, among other materials that are possible, each of which is contemplated herein.

In other embodiments, the oral stimulator(s) 806 on the two wires 804 is formed of and/or include a different material. For example, each oral stimulator 806 on a first wire 804 is formed of and/or includes stainless steel and each oral stimulator 806 on a second wire 804 is formed of and/or includes gold, among other materials that are possible, each of which is contemplated herein.

In still other embodiments, at least one oral stimulator 806 on a first wire 804 is formed of and/or includes the same material as at least one oral stimulator 806 on a second wire 804 or at least one oral stimulator 806 on a first wire 804 is formed of and/or includes a different material than at least one oral stimulator on a second wire 804. For example, at least one oral stimulator 806 on a first wire 804 and at least one oral stimulator 806 on a second wire 804 are formed of and/or include a precious metal and/or at least one oral stimulator 806 on a first wire 804 is formed of and/or includes a precious metal and at least one oral stimulator 806 on a second wire 804 is formed of and/or includes a base metal, among other materials that are possible, each of which is contemplated herein.

In various embodiments including a plurality of wires 804 and especially three (3) or more wires, each wire 804 may include any suitable quantity of oral stimulators 806, which may be the same quantity and/or a different quantity than one or more other wires 804. In some embodiments, the quantity of oral stimulators 806 may sequentially increase away from the body 802 such that the smallest quantity of oral stimulators 806 is located on the wire 804 closest to the body 802 (e.g., wire 804C) and the largest quantity of oral stimulators 806 is located on the wire 804 farthest from the body 802 (e.g., wire 804A). In alternative embodiments, the quantity of oral stimulators 806 may sequentially decrease from the body 802 such that the largest quantity of oral stimulators 806 is located on the wire 804 closest to the body 802 (e.g., wire 804C) and the smallest quantity of oral stimulators 806 is located on the wire 804 farthest from the body 802 (e.g., wire 804A). In other alternative embodiments, the largest quantity of oral stimulators 806 is located on the wire 804 midway from the body 802 (e.g., wire 804B).

In some embodiments, each wire 804 may include the same shaped oral stimulators 806 thereon. In other embodiments, at least two wires 804 include one or more of the same shaped oral stimulators 806 thereon and/or at least two wires 804 include one or more different shaped oral stimulators 806 thereon.

In additional or alternative embodiments, each wire 804 may include the same sized oral stimulators 806 thereon and/or at least two wires 804 include one or more of the same sized oral stimulators 806 thereon and/or at least two wires 804 include one or more different sized oral stimulators 806 thereon. In some embodiments, the size of the oral stimulator(s) 806 may sequentially increase away from the body 802 such that the smallest sized oral stimulator 806 is located on the wire 804 closest to the body 802 (e.g., wire 804C) and the largest sized oral stimulator 806 is located on the wire 804 farthest from the body 802 (e.g., wire 804A). In alternative embodiments, the size of the oral stimulator(s) 806 may sequentially decrease from the body 802 such that the largest sized oral stimulator 806 is located on the wire 804 closest to the body 802 (e.g., wire 804C) and the smallest sized oral stimulator 806 is located on the wire 804 farthest from the body 802 (e.g., wire 804A). In other alternative embodiments, the largest sized oral stimulator 806 is located on the wire 804 midway from the body 802 (e.g., wire 804B).

In some embodiments, each wire 804 may include oral stimulators 806 thereon including the same material(s). In other embodiments, at least two wires 804 include one or more oral stimulators 806 thereon including the same material(s) and/or at least two wires 804 include one or more oral stimulators 806 thereon including different material(s).

Handle 808 may include any suitable shape and/or materials that can facilitate handling of the oral device 800. In some embodiments, the handle 808 can be used to assist in inserting and removing the oral device 800 from the oral cavity of a user. At least in the illustrated embodiment, the handle 808 includes a U-shape so that the oral device 800 can be attached to an external object (e.g., a necklace) via the handle 808 so that the oral device is not dropped and/or lost when disengaged from the user's oral cavity.

Referring to FIG. 9A, one embodiment of the various dimensions of the oral device 800 are illustrated thereon. At least in the illustrated embodiment, the oral device 800 and/or body 802 includes a width A in the range of about 24 mm to about 32 mm, among other sizes and ranges of sizes that are possible, each of which is contemplated herein. In one embodiment, the oral device 800 includes a width A of about 28 mm, among other sizes that are possible, each of which is contemplated herein.

The body 802, in various embodiments, includes a length B in the range of about 12 mm to about 20 mm, among other sizes and ranges of sizes that are possible, each of which is contemplated herein. In one embodiment, the body 802 includes a length B of about 16.5 mm, among other sizes that are possible, each of which is contemplated herein.

The body 802, in various embodiments, includes a length B in the range of about 12 mm to about 20 mm, among other sizes and ranges of sizes that are possible, each of which is contemplated herein. In one embodiment, the body 802 includes a length B of about 16.5 mm, among other sizes that are possible, each of which is contemplated herein.

The oral device includes a length C that extends from the handle 808 to a ring 804C that is closest to the body 802. In various embodiments, the length C in the range of about 30 mm to about 40 mm, among other sizes and ranges of sizes that are possible, each of which is contemplated herein. In one embodiment, the length C is about 35 mm, among other sizes that are possible, each of which is contemplated herein.

The oral device includes a length D that extends from the handle 808 to a ring 804B that is midway from the body 802. In various embodiments, the length D in the range of about 35 mm to about 45 mm, among other sizes and ranges of sizes that are possible, each of which is contemplated herein. In one embodiment, the length D is about 41.5 mm, among other sizes that are possible, each of which is contemplated herein.

The oral device includes a length E that extends from the handle 808 to a ring 804C that is farthest from the body 802. In various embodiments, the length E in the range of about 43 mm to about 62 mm, among other sizes and ranges of sizes that are possible, each of which is contemplated herein. In one embodiment, the length E is about 48 mm, among other sizes that are possible, each of which is contemplated herein.

In FIG. 9B, other exemplary dimensions of the oral device 800 are illustrated. The body 802, in various embodiments, includes a minimum thickness F in the range of about 2 mm to about 4 mm, among other sizes and ranges of sizes that are possible, each of which is contemplated herein. In one embodiment, the thickness F is about 3 mm, among other sizes that are possible, each of which is contemplated herein.

In some embodiments, the body 802 includes a maximum thickness G in the range of about 8 mm to about 12 mm, among other sizes and ranges of sizes that are possible, each of which is contemplated herein. In one embodiment, the thickness G is about 10 mm, among other sizes that are possible, each of which is contemplated herein.

As shown in FIG. 9B, the length H of the body 802, in various embodiments, is in the range of about 15 mm to about 25 mm, among other sizes and ranges of sizes that are possible, each of which is contemplated herein. In one embodiment, the length H is about 19 mm, among other sizes that are possible, each of which is contemplated herein.

Here, the length I of the thickness G of the body 802 is in the range of about 3 mm to about 7 mm, among other sizes and ranges of sizes that are possible, each of which is contemplated herein. In one embodiment, the length I is about 5 mm, among other sizes that are possible, each of which is contemplated herein.

In various embodiments, a length J that the shape of the wire 804A extends from the body 802 is in the range of about 15 mm to about 25 mm, among other sizes and ranges of sizes that are possible, each of which is contemplated herein. In one embodiment, the length J is about 19.5 mm, among other sizes that are possible, each of which is contemplated herein.

The handle 808, in various embodiments, includes a length K. In various embodiments, the length K in the range of about 8 mm to about 12 mm, among other sizes and ranges of sizes that are possible, each of which is contemplated herein. In one embodiment, the length K is about 10 mm, among other sizes that are possible, each of which is contemplated herein.

In various embodiments, it is recommended that the various embodiments be used for approximately fifteen (15) minutes, three times (3x) a day. It is recommended that the various embodiments discussed herein not be used all day and/or for extended periods of time.

In the above description, certain terms may be used such as “receiving,” “inserting,” “attaching,” “connecting,” and the like. These terms are used, where applicable, to provide some clarity of relationships. But, these terms are not intended to be absolute relations. Rather, these terms may be interchangeable with one another.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other but including one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

In the above description, certain terms may be used such as “up,” “down,” “upper,” “lower,” “horizontal,” “vertical,” “left,” “right,” “over,” “under,” and the like. These terms are used, where applicable, to provide some clarity of description when dealing with relative relationships. However, these terms are not intended to imply absolute relationships, positions, and/or orientations. For example, with respect to an object an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.” Moreover, unless otherwise noted, as defined herein a plurality of particular features does not necessarily mean every particular feature of an entire set or class of the particular features.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

In addition, as used herein, the term, “set,” can mean one or more, unless expressly specified otherwise. The term, “sets,” can mean multiples of or a plurality of one or mores, ones or more, and/or ones or mores consistent with set theory, unless expressly specified otherwise.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The present subject matter may be embodied in other specific forms without departing from its spirit or essential characteristics. The described examples are to be considered in all respects only as illustrative and not restrictive. All changes which come within the meaning and range of equivalency of the claims are to be embraced within their scope.

The subject matter of the present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An oral device, comprising:
a body configured for insertion into an oral cavity of a user;
a first wire coupled to a first point on the body and extending away from the body and returning to couple to a second point on the body located apart from the first point to form a first wire shape and a first space between the first wire and the body; and
a first set of one or more oral stimulators coupled to and movable on the first wire.

2. The oral device of claim 1, wherein the first set of one or more oral stimulators includes a plurality of oral stimulators.

3. The oral device of claim 2, wherein each oral stimulator in the plurality of oral stimulators includes a shape comprising one of a sphere, an ovoid, an ellipsoid, a tetrahedron, a pyramid, a cube, or a cuboid.

4. The oral device of claim 2, wherein each oral stimulator in the plurality of oral stimulators includes a same shape.

5. The oral device of claim 2, wherein each oral stimulator in the plurality of oral stimulators includes one of a same shape or a different shape.

6. The oral device of claim 2, wherein at least two oral stimulators in the plurality of oral stimulators include a different shape.

7. The oral device of claim 2, wherein at least two oral stimulators in the plurality of oral stimulators include a different size.

8. The oral device of claim 1, wherein at least one oral stimulator in the first set of one or more oral stimulators is one of movable along the first wire, rotational on the first wire, or movable along and rotational on the first wire.

9. The oral device of claim 1, further comprising:
a second wire coupled to a third point on the body and extending away from the body and returning to couple to a fourth point on the body located apart from the third point to form a second wire shape and a second space between the second wire and the body; and
a second set of one or more oral stimulators coupled to and movable on the second wire.

10. The oral device of claim 9, wherein the second set of one or more oral stimulators includes a plurality of oral stimulators.

11. The oral device of claim 10, wherein each oral stimulator in the plurality of oral stimulators includes a shape comprising one of a sphere, an ovoid, an ellipsoid, a tetrahedron, a pyramid, a cube, or a cuboid.

12. The oral device of claim 11, wherein each oral stimulator in the plurality of oral stimulators includes a same shape.

13. The oral device of claim 11, wherein each oral stimulator in the plurality of oral stimulators includes a different shape.

14. The oral device of claim 11, wherein at least two oral stimulators in the plurality of oral stimulators include a different shape.

15. The oral device of claim 9, wherein at least one oral stimulator in the second set of one or more oral stimulators is one of movable along the second wire, rotational on the second wire, or movable along and rotational on the second wire.

16. The oral device of claim 9, wherein:
the first set of one or more oral stimulators includes a first plurality of oral stimulators;
the second set of one or more oral stimulators includes a second plurality of oral stimulators; and
the first plurality of oral stimulators and the second plurality of oral stimulators include one of a same quantity of oral stimulators or a different quantity of oral stimulators.

17. The oral device of claim 16, wherein:
at least one oral stimulator in the first plurality of oral stimulators includes a different shape from at least one oral stimulator in the second plurality of oral stimulators.

18. The oral device of claim 16, wherein:
at least one oral stimulator in the first plurality of oral stimulators includes a different size than at least one oral stimulator in the second plurality of oral stimulators.

19. An oral device, comprising:
a body configured for insertion into an oral cavity of a user, the body including:
a space for accommodating one or more teeth of a user;
an exterior wall;
an engagement wall;
a first side coupled to the exterior wall and configured for engagement by lips or one or more teeth of the user; and
an interior wall coupled to a second side of the engagement wall,
wherein:
the interior wall is opposite the exterior wall,
the interior wall comprises an open space,
the open space comprises at least one connection for a ball assembly, and
the ball assembly is located within the open space of the interior wall.

20. An oral device, comprising:
a member configured to define at least one space for accommodating one or more teeth of a user, the member comprising:
an engagement wall, configured to be engaged by lips or one or more teeth of a user; and
an interior wall coupled to the engagement wall,
wherein:
the interior wall comprises an open space,
the open space comprises at least one connection for a ball assembly, and
the ball assembly is located within the open space of the interior wall.
